(19) United States
(12) Patent Application Publication
(10) Pub. No.: US 2009/0177132 A1
(43) Pub. Date: Jul. 9, 2009

(54) Inflatable Splint

(75) Inventors: Daniel REIS, Haifa (IL); Dalia ZUCKER, Haifa (IL); Asher Bin NUN, Carmiel (IL)

Correspondence Address: HOUSTON ELISEEVA, 4 MILITIA DRIVE, SUITE 4, LEXINGTON, MA, 02421, US

(73) Assignee: ORTHO-FLEX LTD., Haifa (IL)

(21) Appl. No.: 12/403,978

(22) Filed: Mar. 13, 2009

Related U.S. Application Data
(63) Continuation of application No. PCT/IL2007/001111, filed on Sep. 10, 2007.
(60) Provisional application No. 60/844,383, filed on Sep. 14, 2006.

(51) Int. Cl. A61F 5/00

(52) U.S. Cl. 602/13; 602/27

(57) ABSTRACT

An inflatable splint is presented. The splint comprises integral foot-accommodating portion, calf-accommodating portion and intermediate, ankle-accommodating portion. The splint is configured to be fitted over a leg part comprising a foot, ankle and calf such that front faces of the leg part are exposed. The splint has a collapsed state and has an open state in which it can be mounted on the leg part. When opened, the splint assumes the general form of the leg part.

### RELATED APPLICATIONS

This application is a Continuation of International application Number PCT/IL2007/001111, filed on Sep. 10, 2007, which in turn claims the benefit under 35 USC 119(e) of U.S. Provisional Application No. 60/844,383, filed on Sep. 14, 2006, both of which are incorporated herein by reference in their entirety.

### FIELD OF THE INVENTION

The present invention relates to an inflatable splint which may be used, for example, in emergency situations, before and after surgical operations on the limbs, fractures, intensive care, and heel spurs.

### BACKGROUND OF THE INVENTION

A splint is a medical device for the immobilization of limbs. It can be used by the emergency medical services or by volunteer first responders, to immobilize a fractured limb before the transportation; it is then a temporary immobilization. Splints can also be used by nurses and physicians in an emergency room, operation room, intensive care unit, or by a physiotherapist, via a physician's order, to support or immobilize an articulation (e.g. the ankle).

As many injuries and fractures to legs occur in locations that are distant from immediate, competent medical attention, and transportation of the injured person may be hazardous and painful resulting in further injury to the fractured leg, the splint should meet such requirements as size, ease of application and protection of the injured limb, to be capable of being folded into a compact small size that can fit into a first-aid kit to be easily carried in an ambulance or the like, and to be capable of being properly applied by a person other than a medical doctor and without further injury or damage to the broken leg. Various such splint devices have been developed. Examples of inflatable splint devices are disclosed in U.S. Pat. No. 5,957,872, and in co-assigned PCT Application WO 2005/084136.

### SUMMARY OF THE INVENTION

The present invention is concerned with a new inflatable splint that includes novel features over those of known inflatable splints. The splint of the present invention is useable for fixation or support of a leg part. The leg part includes at least the lower part of the leg (“lower leg”) including at least the foot, the ankle and the calf, or the whole leg up to above the knee.

The inflatable splint of the invention includes either one continuous air pocket or a plurality of air pockets (tubes) that can be inflated, whereupon the splint assumes a cradle form for the lower limb in which it is relatively stiff, and can optionally assume variable degrees of stiffness to be used as a leg fixation or support. The stiffness of the brace is varied according to the pressure of the inflation. When folded before inflation, the splint is a small flat package.

In the following, some embodiments of the invention are described. These embodiments may be applied separately or each embodiment may be applied in combination with one or more of the other defined embodiments.

In accordance with one embodiment of the invention the splint is configured to have a collapsed or folded state and an open state and once opened the splint assumes the general form of the leg part. This is of advantage as once opened the splint is an immediate configuration for mounting on the leg. The splint may be inflated before or after mounting. Also, at times the splint may be partially inflated before mounting and then inflated to a final desired degree, e.g. based on medical considerations, after mounting.

In accordance with this embodiment there is thus provided an inflatable splint comprising integral foot-accommodating portion, a calf-accommodating portion and an intermediate, ankle-accommodating portion; the splint being configured to be fitted over a leg part comprising a foot, ankle and calf such that front faces of the leg part are exposed; the splint having a collapsed state and having an open state in which it can be mounted on the leg part; when opened the splint assumes the general form of the leg part.

As indicated above, the splint assumes the general form of the leg part (at least lower leg) once opened, both in its inflated and non-inflated state.

In accordance with another embodiment of the invention the splint is provided with a rigid foot floor-support panel and a foot cushion disposed above the panel. This imparts rigid support for the foot and more evenly distributed pressure on the foot, on the other hand.

In accordance with this embodiment there is thus provided an inflatable splint comprising integral foot-accommodating portion, a calf-accommodating portion and an intermediate, ankle-accommodating portion, the splint being configured to be fitted over a leg part comprising a foot, ankle and calf such that front faces of the leg are exposed; said foot-accommodating portion has a rigid floor-support panel and a foot cushion, typically although not exclusively, inflatable, disposed above said panel.

In accordance with another embodiment of the invention the splint is provided with adjustable fasteners which have one of their ends connected to the side of one of the splint's portions in a releasable arrangement. This permits rapid mounting of the splint over a patient's leg part.

In accordance with this embodiment there is thus provided an inflatable splint comprising integral foot-accommodating portion, a calf-accommodating portion and an intermediate, ankle-accommodating portion; the splint being configured to be fitted over a leg part comprising a foot, ankle and calf such that front faces of the leg part are exposed; the splint portions having side surfaces that in use are disposed over sides of the leg part with edges that leave the front faces exposed; said side surfaces being provided with one or more first adjustable fasteners that link opposite side surfaces to one another; one end of the first fasteners being linked to an anchor member that is connected in a releasable arrangement to one of the side surfaces.

The releasable arrangement may include a zipper but may also include other types of arrangement such as that which includes a hook-and-pile couple (e.g. one known as “Velcro®”) or any kind of quick release buckle, one of which is on a member connected to said end of the fastener arrangement and the other to said one of the sides surfaces.

The first fasteners, in the above embodiment, are typically disposed on side surfaces of the calf-accommodating portion. In some embodiments of the invention, one or more second fasteners are provided that link opposite side surfaces of the foot-accommodation portion.

In accordance with another embodiment of the invention a novel outrigger stabilizer member is provided that can be attached to the splint in a detachable manner for inhibiting axial rotation of a fixed patient's leg. The outrigger attachment controls the limb axis rotation.

In accordance with this embodiment there is thus provided an inflatable splint comprising integral foot-accommodating portion, a calf-accommodating portion and an intermediate, ankle-accommodating portion; the splint being configured to be fitted over a leg part comprising a foot, ankle and calf such that front faces of the leg part are exposed; the splint having a collapsed state and having an open state in which it can be mounted on the leg part; the splint being provided with an outrigger stabilizer member configured for attachment in a detachable manner to a bottom face of the foot-accommodating portion and having an edge that once connected is positioned at the rear of the foot-accommodating portion, and projects laterally from the splint.

A typical detachable arrangement is through a hook-and-pile couple arrangement. Typically, said outrigger stabilizer member has an attachment surface that extends lengthwise over a substantial part of the bottom face. For example, the attachment surface of the outrigger stabilizer member may be fitted with one member of the hook-and-pile couple, and the other member of said couple being disposed on said bottom face. According to some embodiments of the invention, the outrigger stabilizer member comprises a tongue that extends rearwards from said edge and is configured for attachment to an attachment surface in the rear face of the ankle-accommodating portion of the splint.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIGS. 1-3 shows a splint in accordance with an embodiment of the invention in which FIG. 1 shows the splint in a closed state, FIG. 2 shows the splint, in a non-inflated state, and FIG. 3 shows the splint in an inflated state.

FIG. 4A is a partial longitudinal cross-section through the lower parts of the splint according to an embodiment of the invention, open and in a non-inflated state.

FIG. 4B is a partial cross-section through the lower part of a splint fitted with a heel raise pad.

FIG. 4C illustrates an inflatable splint according to an embodiment of the invention having a heel raise pad and pad extensions along the sides of the inner surface of the foot-accommodating portion.

FIG. 5 is a partial transverse cross-section through the lower part of the splint of FIG. 4A, in an inflated state.

FIG. 6 is a splint according to an embodiment of the invention in which the bottom of the foot-accommodating portion has a pocket for holding a folded outrigger stabilizer member shown removed from the pocket.

FIG. 7 shows the outrigger in an open, unfolded state.

FIG. 8 is a side perspective view of a splint, in an inflatable state, fitted with an outrigger member.

FIG. 9 shows a view from the direction of arrow XI in FIG. 8.

### DETAILED DESCRIPTION OF THE PREFERRED EMBODIMENTS

Exemplary embodiments of the invention are shown in and described with reference to FIGS. 1-9. As will be appreciated, these embodiments while being shown for purpose of illustration of the various features of the invention as claimed herein, is non-limiting and various modifications of this embodiment, as will be clear to a person versed in the art, are possible.

Shown in FIGS. 1-3 is a splint generally designated as 20 having a body including a calf-accommodating portion 22, a foot-accommodating portion 24 integrally formed and linked together through an intermediate ankle-accommodating portion 26. In the closed state shown in FIG. 1, the splint is folded about the heel part of the ankle-accommodating portion 26 to assume a relatively compact configuration of a small flat package in which it can be stored until use.

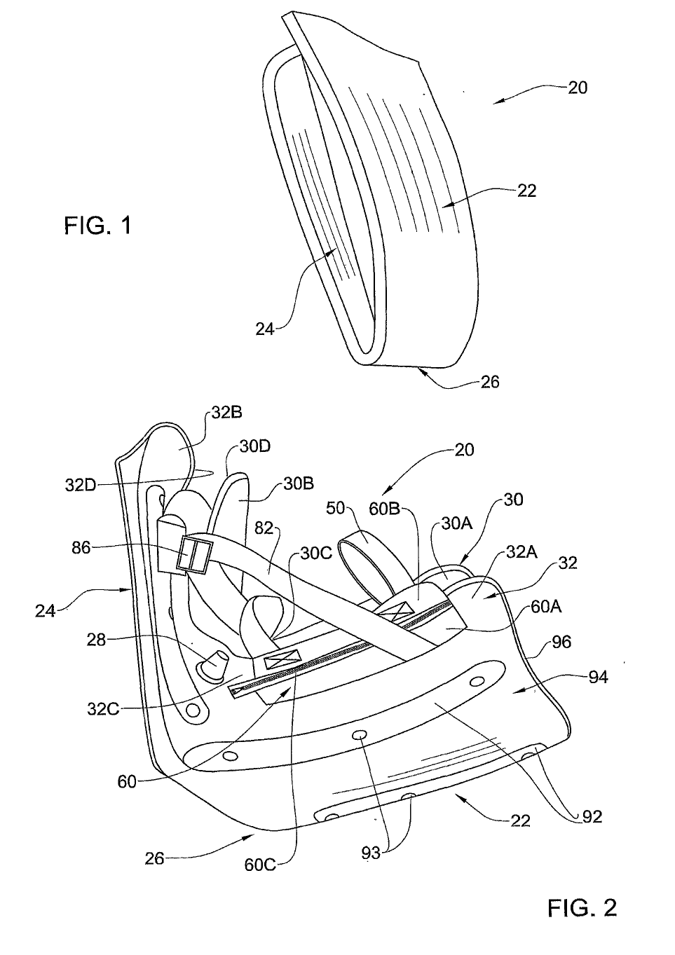

As can be seen in FIG. 2, the splint includes a filling nozzle to permit introduction of inflation fluid, which may be a liquid or a gas, typically air. The advantage of using a gas is that even while fully inflated, inflated pockets in the splint, to be described below, will have some residual flexibility and a cushion effect. A liquid as an inflation fluid may be useful, for example, for applications in which a cooling effect is desired. Furthermore, the filling with a liquid will render the inflated splint to be heavier than the case of a gas, such as air, such weight being of advantage for one application while light weight of a gas-inflated splint being advantageous for others.

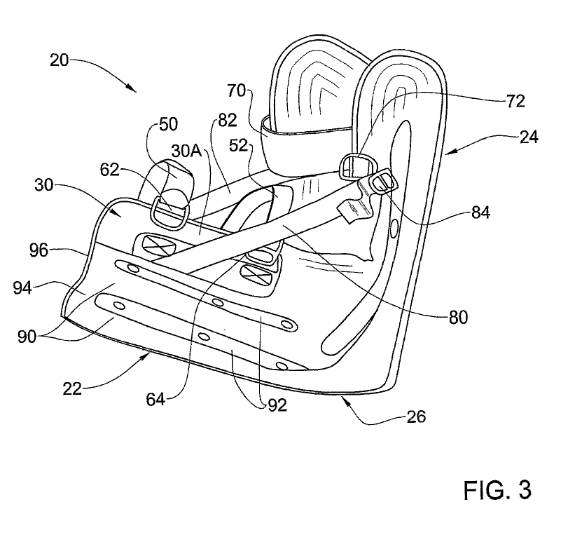

In FIG. 2 the splint is shown in an open, yet non-inflated state. As can be seen in this figure, once opened the splint has already a general boot-like form in which in can be fitted over the leg part (lower leg in the present example). In this form the calf-accommodating portion 22 is essentially normal to the foot-accommodating portion 24. Upon inflation with a fluid, typically, although not exclusively, air, as shown in FIG. 3, the splint becomes rigid. In an inflated state the fluid containing pockets of the splint expand outwards and also towards the splint interior whereby they create a supporting cradle for the subject's leg.

As can be seen in FIGS. 2 and 3, the splint is formed with side surfaces that include a right side surface 30 and a left side surface 32, each of which being provided with respectable first side surfaces 30A and 32A, which can fit over the sides of a subject's calf, second respective side surfaces 30B and 32B, which are intended to fit over sides of a foot and intermediate respective side surfaces 30C and 32C, fitting over sides of the ankle. The side surfaces have respective edges 30D and 32D that define therebetween an open space for exposure of front faces of the leg part.

The splint shown in FIGS. 1-3 is provided, in this specific embodiment, with a number of inflatable tubes, in fluid communication with one another, such as that disclosed in WO 2005/084136, the content of which is incorporated herein by reference. Thus, all air pockets can be inflated through fluid ingress through nozzle 28. As will be appreciated the position of the nozzle 28 is but an example and the nozzle may be positioned in any suitable place in the splint.

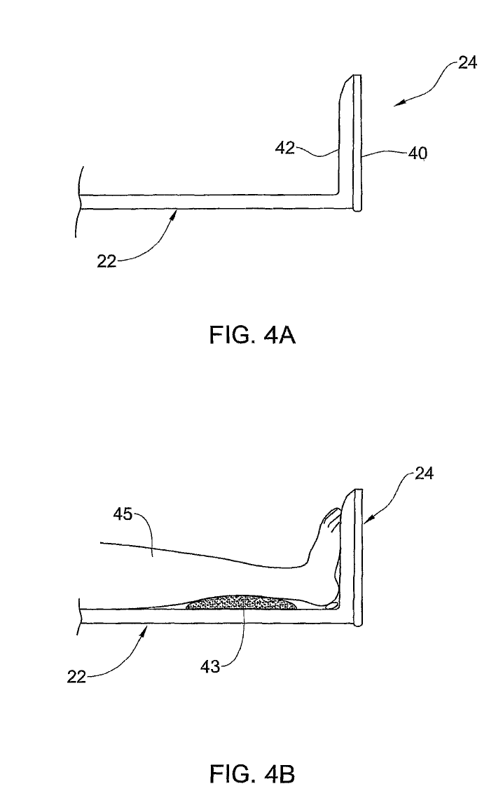

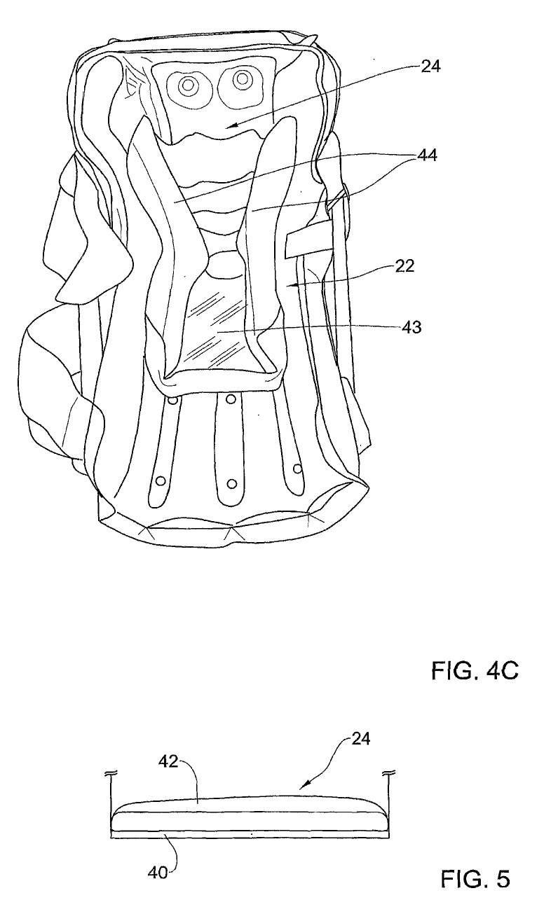

Reference is now being made to FIGS. 4A and 5, where FIG. 4A is a partially longitudinal cross-section of the splint in a non-inflated state and FIG. 5 is a transverse cross-section through the sole portion (foot-accommodating portion) 24 in an inflated state of the same splint. As can be seen in FIGS. 4A and 5, the foot-accommodating portion 24 is provided with a rigid floor support panel 40 overlaid with a foot cushion 42 which in accordance with one embodiment of the invention is inflatable. However, it should be noted that the foot cushion may be formed also, for example, from a flexible, non-inflatable matrix, typically covered or completely enclosed within a sheet which may be made of a cloth, liquid impermeable polymer, etc.

As can be seen in FIGS. 4B and 4C, the splint may also be provided with an optional heel raise pad 43, placed immediately behind and bordering on the special heel depression in the nylon of the heel portion, and possibly also pad extensions 44 along the sides of the inner surface of the foot-accommodating portion 24 which protect the heel and the borders of the foot from pressure injury. The heel raise pad (e.g. with its extensions) may be inflatable or may be made from a soft flexible non-inflatable matrix. In FIG. 4B, pad 43 is shown as raising the heel part in lower leg 45. In accordance with one embodiment the pad 43 is fixed within the splint. In accordance with another embodiment the pad is removable.

As can be seen in FIGS. 2 and 3, the first side surfaces 30A and 32A are connectable to one another through two length-adjustable straps 50 and 52. It should be appreciated that the adjustable straps are one of many examples of fasteners that may be used for connecting the two side surfaces to one another, such as for example, strings, laces and others. Furthermore, as can be appreciated, the number of straps or other fasteners may be different than two; there may be one fastener according to some embodiment or a plurality, more than two fasteners according to others.

Fasteners 50 and 52 are fixed at one of their ends to an anchor member 60, which is secured at side surface 32A and connected at their other end to side surface 30A through buckles 62 and 64. In use, the straps 50 fit over the front face of a subject's calf and ankle and serve to secure the splint in position.

Anchor member 60 is a two part member consisting of parts 60A and 60B releasably connected to one another through zipper 60C. Through opening of zipper 60C the part 60B with the associated straps can be released to permit easy fitting of the splint onto a subject's leg and then after fitting rapid securing of the straps over the subject's front leg surfaces. The rapid application arrangement, such as one comprising a zipper 60C is especially important in long leg versions of the splint. The straps 50 and 52 are connected and are length adjustable through respective buckles 62 and 64. As will be appreciated a buckle arrangement is one of many length adjustable fastening arrangements known per se.

It should be noted that the use of a zipper is only one option for releasable anchor arrangement and other arrangements such as that which make use of a hook and pile couple, are possible.

Optionally, the splint is provided also with an adjustable fastening arrangement for linking second side surfaces 30B and 32B. As can be seen in FIG. 3, provided is a strap 70 that links the two side surfaces which is fixed to side surface 32B and adjustably connected through buckle 72 to side surface 30B.

Another feature according to a preferred embodiment of the invention is an adjustable strap that links the foot-accommodating portion and the calf-accommodating portion for dorsiflexing of the ankle joint. This can be seen in FIGS. 2 and 3 where straps 80 and 82 are provided which are fixed to the upper part of first side surfaces 30A and 32A and are adjustably fitted through respective buckles 84 and 86 to side surfaces 30B and 32B. Straps 80 and 82 are operative to maintain the ankle at neutral position, e.g. about 90 degrees, or when desired they can be tightened to dorsiflex the ankle creating tension in the heel cord and the plantar fascia of the sole of the foot.

Typically, the splint body is formed from two parallel polymeric sheets and the air pockets within the splint are formed through a series of spaced apart welds that define inflatable air pockets 90 between them, separated by non-inflatable portions, such as portions 92. These non inflatable portions are perforated by a series of ventilation holes 93. In accordance with an embodiment of the invention the welds are designed such as to provide a continuous inflatable portion 94 adjacent the splint's upper rim 96. Preferably, as can be seen also in the embodiment herein, the continuous inflatable portion adjacent to the rim also extends along rims of the side portion 30.

The body may be made of any suitable weldable material of a kind that can withstand the required pressure of the fluid within the inflatable portions without rupturing. In some embodiments of the invention the gas pressure is around 0.25 atmospheres and may reach as high as 1 atmosphere without leakage of gas. In accordance with some embodiments of the invention, the splint body is made of nylon coated with polyurethane.

The fasteners may be fixed to the splint body through a number of arrangements, among others depending on the material of the splint body and that of the fasteners. In the specific embodiment where the splint body is made from a polymeric material and the straps from a woven material, a typical, but not exclusive, manner of securing may be by sewing.

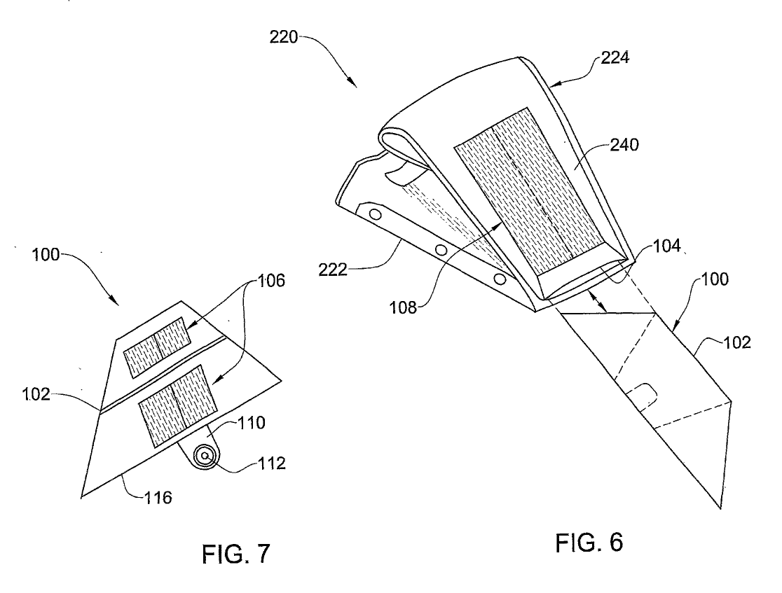

FIGS. 6-9 show an embodiment of the invention in which use is made of an outrigger stabilizer member 100. In FIGS. 6, 8 and 9 like reference numerals to those of FIGS. 1-3 are used, shifted by 200 to designate similar components. In the shown example, the outrigger member 100 is substantially rigid and is foldable about line 102 into a folded state as can be seen in FIG. 6. In FIG. 6, splint 220 is shown in a partial folded state. Splint 220 seen in FIG. 6 is provided with a pocket with a rear-end opening 104, formed in the bottom of the foot-accommodating portion 222. In a typical example, the pocket is a lengthwise space below the floor-support panel 240.

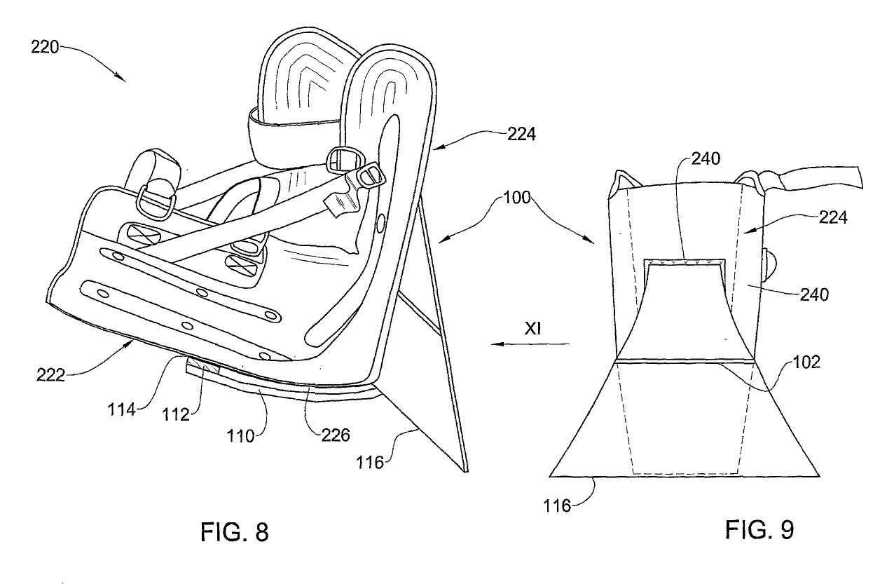

The outrigger member 100, has in the shown exemplary embodiment, and overall trapezoidal shape and is provided with and attachment device 106 which is designed for attachment to a corresponding attachment device 108 at the bottom of the leg accommodating portion 222 of the splint 220. By a specific example, devices 106 and 108 are a hook-and-pile couple. The outrigger member 100 is preferably provided with a tongue 110, extending from the rear edge 116, having an attachment device 112, for attaching to a corresponding attachment device 114, in the rear face of the ankle-accommodating portion 226 shown in FIG. 8.

FIGS. 8 and 9 show the outrigger in its operative position being attached to the bottom face of the foot-accommodating portion with its rear edge 116, being essentially aligned with the heel part of the ankle-accommodating portion 226 or slightly projecting in a rearward direction from the rear end of the bottom of the foot-accommodating portion (e.g. a 3-5 cm), and projecting laterally from the two sides of the splint. The tongue member 110 prevents the outrigger 100 from being torn off when the leg is moved, particularly when the leg is drawn up by flexing the thigh. The outrigger stabilizes the whole leg in neutral rotation and prevents the leg from rolling out or inwards. This is of particular importance in injuries of the thigh and hip.

The embodiment shown herein is designed for supporting the leg and the ankle. As will be appreciated, the invention also applies to inflatable splints for different purposes, such as one intended to support an entire leg and parts of or the whole of the upper limb.

While this invention has been particularly shown and described with references to preferred embodiments thereof, it will be understood by those skilled in the art that various changes in form and details may be made therein without departing from the scope of the invention encompassed by the appended claims.

What is claimed is:

1. An inflatable splint comprising integral foot-accommodating portion, a calf-accommodating portion and an intermediate, ankle-accommodating portion; the splint being configured to be fitted over a leg part comprising a foot, ankle and calf such that front faces of the leg part are exposed; the splint having a collapsed state and having an open state in which it can be mounted on the leg part; when opened the splint assumes the general form of the leg part.

2. The inflatable splint according to claim 1, which assumes the general form of the leg part in both inflated and non-inflated state.

3. The inflatable splint according to claim 1, wherein said foot-accommodating portion has a rigid floor-support panel and a foot cushion disposed above said panel.

4. The inflatable splint according to claim 3, wherein said foot cushion is inflatable.

5. The inflatable splint according to claim 3, comprising a pocket defined under the foot panel for accommodating at least one splint accessory.

6. The inflatable splint according to claim 5, wherein said accessory is an outrigger stabilizer for attachment to the splint to hinder axial rotation of the leg.

7. The inflatable splint according to claim 1, wherein the splint portions have side surfaces that in use are disposed over sides of the leg part with edges that define therebetween the exposed front faces, said side surfaces being provided with one or more first adjustable fasteners that link opposite side surfaces to one another; one end of the first fasteners being linked to an anchor member that is connected in a releasable arrangement to one of the side surfaces.

8. The inflatable splint according to claim 7, wherein said releasable arrangement is a zipper or a hook-and-pile couple or a quick release buckle.

9. The inflatable splint according to claim 7, wherein said first fasteners are disposed on side surfaces of the calf-accommodating portion.

10. The inflatable splint according to claim 9, comprising one or more second fasteners that link opposite side surfaces of the foot-accommodating portion.

11. The inflatable splint according to claim 6, wherein said outrigger stabilizer member is configured to be attachable to the splint in a detachable manner, and configured for attachment to a bottom face of the foot-accommodating portion, the outrigger stabilizer member having an edge that once connected is position at the rear of the foot-accommodating portion and projects laterally from the splint.

12. The inflatable splint according to claim 11, wherein the detachable attachment is through a hook-and-pile couple arrangement.

13. The inflatable splint according to claim 12, wherein said outrigger stabilizer member has an attachment surface that extends lengthwise over a substantial part of said bottom face.

14. The inflatable splint according to claim 11, wherein said outrigger stabilizer member comprises a tongue extending rearwards from said edge and being configured for attachment to an attachment surface in a rear face of the splint.

15. The inflatable splint according to claim 11, wherein said outrigger stabilizer member is foldable.

16. The inflatable splint according to claim 15, comprising a pocket for accommodating said outrigger stabilizer member.

17. The inflatable splint according to claim 1, comprising adjustable straps between the foot-accommodating portion and the ankle portion for dorsiflexing the ankle joint.

18. The inflatable splint according to claim 1, comprising inflatable and non-inflatable portions, said inflatable portions being designed to withstand pressures of up to 1 atmosphere without gas leakage or bursting.

19. The inflatable splint according to claim 18, wherein at least said inflatable portions are made of nylon coated with polyurethane.

20. The inflatable splint according to claim 1, comprising an inflatable cushioned heel raise support pad.

21. The inflatable splint according to claim 20, comprising foot border protection pads.

22. The inflatable splint according to claim 20, wherein the pad is non-inflatable.

23. The inflatable splint according to claim 20, wherein the pad is fixed within the splint.

24. The inflatable splint according to claim 20, wherein the pad is removable.

25. The inflatable splint according to claim 1, comprising inflatable and non-inflatable portions, said inflatable portions defining a substantially continuous inflatable belt adjacent an edge of the splint.

26. The inflatable splint according to claim 23, wherein said inflatable portions define a substantially continuous inflatable belt adjacent an upper edge of the splint.

27. An inflatable splint comprising integral foot-accommodating portion, a calf-accommodating portion and an intermediate, ankle-accommodating portion, the splint being configured to be fitted over a leg part comprising a foot, ankle and calf such that front faces of the leg part are exposed; said foot-accommodating portion having a rigid floor-support panel and a foot cushion disposed above said panel.

28. An inflatable splint comprising integral foot-accommodating portion, a calf-accommodating portion and an intermediate, ankle-accommodating portion; the splint being configured to be fitted over a leg part comprising a foot, ankle and calf such that front faces of the leg part are exposed; the splint portions having side surfaces that in use are disposed over sides of the leg part with edges that define therebetween the exposed front faces; said side surfaces being provided with one or more first adjustable fasteners that link opposite side surfaces to one another; one end of the first fasteners being linked to an anchor member that is connected in a releasable arrangement to one of the side surfaces.

29. An inflatable splint comprising integral foot-accommodating portion, a calf-accommodating portion and an intermediate, ankle-accommodating portion; the splint being configured to be fitted over a leg part comprising a foot, ankle and calf such that front faces of the leg part are exposed; the splint having a collapsed state and having an open state in which it can be mounted on the leg part; the splint being provided with an outrigger member attachable to the splint in a detachable manner, the outrigger member configured for attachment to a bottom face of the foot-accommodating portion and having an edge that once connected is position at the rear of the foot-accommodating portion, and projects laterally from the splint.
